(19) United States
(12) Patent Application Publication
(10) Pub. No.: US 2003/0092682 A1
(43) Pub. Date: May 15, 2003

(54) Use of doxycycline for treatment of certain skin and mouth ailments

(76) Inventor: Gary V. Heesch, Salt Lake City, UT (US)

Correspondence Address: Foster & Foster LLC, Mr. Lynn G. Foster, 602 E. 300 S., Salt Lake City, UT, 84102, US

(21) Appl. No.: 09/910,317

(22) Filed: Jul. 20, 2001

(51) Int. Cl. A61K 31/65; A61K 31/59; A61K 31/549

(52) U.S. Cl. 514/152; 514/200

(57) ABSTRACT

A method of treating (a) cold sores in and out of the mouth, (b) canker sores, (c) cancer wounds including but not limited to wounds such as those which fail to heal due to chemotherapy and radiation therapy, (d) surgical wounds of all types, (e) diabetes wounds, (f) decubitus ulcers, (g) athletes foot including chronic athletes foot and (h) scarring with doxycycline and/or cefaclor is disclosed.

### FIELD OF THE INVENTION

The present invention relates generally to the treatment of skin and mouth disorders and, more particularly, to the use of doxycycline and/or cefaclor to eradicate or significantly alleviate certain skin disorders.

### BACKGROUND

In the past, it has been difficult and often impossible to eradicate certain skin and mouth disorders, including various types of sores, wounds, skin ulcers, scarring and severe chronic athletes foot and infections.

Most often, if not always, treatments of the past have either failed to cure the disorder or only offered temporary relief followed by reoccurrence of the ailment. The skin disorders in question comprise cold sores in and out of the mouth, canker sores, cancer wounds including surgical wounds (which fail to heal due to chemotherapy and radiation therapy), other types of surgical wounds, diabetes sores, decubitus ulcers, sores, scarring and athletes foot including chronic athletes foot.

The solution (or solutions) to these ailments has constituted a long-term, unsolved problem.

The present invention represents an efficacious solution to the above-identified problem and comprises topical use of doxycycline and/or cefaclor, earlier used internally and to treat acne, to eradicate or greatly alleviate the above-identified skin and mouth problems.

### BRIEF SUMMARY AND OBJECTS OF THE PRESENT INVENTION

In brief summary, the present invention solves or greatly alleviates skin and mouth ailments, not solved or significantly alleviated by past efforts, by topically treating (a) cold sores in and out of the mouth, (b) canker sores, (c) cancer wounds including but not limited to wounds such as those which fail to heal due to chemotherapy and radiation therapy, (d) surgical wounds of all types, (e) diabetes wounds, (f) decubitus ulcers (g) athletes foot including chronic athletes foot and (h) scarring with doxycycline and/or cefaclor.

It is a primary object of the present invention to solve or greatly alleviate skin and mouth ailments and thereby solve or substantially solve long term problems of the past.

Another paramount object of this invention is to provide a cure for or substantially cure skin and mouth ailments by topically treating (a) cold sores in and out of the mouth, (b) canker sores, (c) cancer wounds including but not limited to wounds such as those which fail to heal due to chemotherapy and radiation therapy, (d) surgical wounds of all types, (e) diabetes wounds, (f) decubitus ulcers (g) athletes foot including chronic athletes foot and (h) scarring with doxycycline and/or cefaclor.

These and other objects and features of the present invention will be apparent from the detailed description.

### DETAILED DESCRIPTION

The topical application of doxycycline and/or cefaclor to certain skin and mouth sores, wounds and scarred regions has surprisingly proven to be very efficacious, as explained in greater detail below. Doxycycline and cefaclor chemically identify the antibiotics of concern, which, respectfully classified as a tetracycline antibiotic and a second generation cephalosporin antibiotic. The antibiotics may be used separately or together.

It is preferred that the antibiotic be mixed in liquid, such as distilled water for ease of application. While not critical 400mg -600 mg of antibiotic may be mixed with 2.0-2.5 ounces of distilled or purified water, for ease of placement on the affected area of the skin and/or the mouth.

To enhance vaporization of the water after the mixture, small amount of drying agent, such as colloidal silver, may be added to the mixture, which also serves as a preservative.

All persons treated for cold sores responded within twenty-four hours, with complete eradication within seventy-two hours for ninety percent (90%) of patients treated. The remaining ten percent (10%) achieved complete success within ten days.

Examples of patients treated for in and out of the mouth cold sores are contained in Table I below.

TABLE I

| Patient | Gender | Age | Results |
|---|---|---|---|
| A | F | 40 | All patients cleared within three days with no |
| B | F | 25 | side effects or dryness experienced. |
| C | F | 60 | All patients were suffering with chronic con- |
| D | M | 20 | ditions prior to treatment. |

All persons treated for canker sores experienced relief from pain within ten minutes, and total eradication within forty-eight hours.

Examples of patients treated for canker sores are contained in Table II below.

TABLE II

| Patient | Gender | Age | Results |
|---|---|---|---|
| A | M | 65 | Pain relief achieved within 5 minutes of |
| B | M | 58 | application. All four patients cleared within |
| C | M | 30 | three days. |
| D | F | 31 | |

Patients with post surgical wounds, post radiation and/or general trauma skin breaks from other causes were treated. Each experienced scabbing within eight hours, and accelerated healing within seven to fourteen days, depending on the condition.

Examples of patients treated for such wounds are contained in Table III below:

TABLE III

| Patient | Gender | Age | Results |
|---|---|---|---|
| A | F | 30 | Patient “A” had pancreatic cancer with surgical |
| B | F | 62 | wounds that would not heal. Healing began |
| | | | within 72 hours of treatment. |

Patients with diabetes sores that threatened to spread into deep tissue and the bones responded with complete healing within ten days with no signs of deep tissue or bone invasion.

Examples of patients treated for diabetes sores are contained in the Table IV below.

TABLE IV

| Patient | Gender | Age | Results |
|---|---|---|---|
| A | M | 68 | Both patients were diabetic and were treated for |
| B | F | 30 | wounds and sores that healed completely in less |
| | | | than the normal time. Patient “A” avoided |
| | | | amputation of leg. |

In respect to scarring and patients with new injuries or surgery that would normally have scarring, after being treated, had little or no scarring when treated with the antibiotic. Furthermore, persons with old scar tissue from whatever cause experienced significant reduction in noticeable scar tissue within 12 months daily treatment.

Examples of patients treated for scarring are contained in Table V below.

TABLE V

| Patient | Gender | Age | Results |
|---|---|---|---|
| A | F | 45 | Patient “A” experienced severe scarring on |
| B | M | 20 | both arms as the result of an automobile acci- |
| C | | | dent. She could not straighten her arms. After |
| D | | | six months of treatment she had regained al- |
| | | | most normal use of her arms. Like many cystic |
| | | | acne patient “B” experienced severe scarring. |
| | | | After nine months of treatment he experienced |
| | | | a 90% reduction in his scarring. |

Examples of patients treated for chronic athletes foot are contained in Table VI.

TABLE VI

| Patient | Gender | Age | Results |
|---|---|---|---|
| Several | M & F | All ages | Many patients have been treated for severe, |
| | | | chronic athletes foot with a combination of |
| | | | doxycycline and an anti-fungal product. |
| | | | 100% of subjects responded within 4 weeks |
| | | | with no reoccurrence of symptoms. |

All of the preceding conditions were treated with doxycycline or cefaclor mixed in a solution of distilled or purified water with 400 to 600 mg of doxycycline in 2 ounces of water. The medication was applied topically to the affected area morning, afternoon and night.

Once the doxycycline or cefaclor is mixed in water, if not used promptly, it may then be kept refrigerated to extend the life and potency of the antibiotic. Two ounces kept refrigerated has a potency life of approximately 30 to 45 days.

The invention may be embodied in other specific forms without departing from the spirit of the central characteristics thereof. The present embodiments therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by letters patent is:

1. A method of treating skin and mouth disorders selected from the group consisting of (a) cold sores in and out of the mouth, (b) canker sores, (c) cancer wounds including but not limited to wounds such as those which fail to heal due to chemotherapy and radiation therapy, (d) surgical wounds of all types, (e) diabetes wounds, (f) decubitus ulcers, (g) athletes foot and (h) scarring with doxycycline and/or cefaclor, comprising the acts of:
placing doxycycline and/or cefaclor in liquid form;
topically applying the liquid directly to a region comprising the disorder.

2. A method according to claim 1 wherein the placing act comprises mixing the doxycycline and/or cefaclor with distilled or purified water.
